(19) United States
(12) Patent Application Publication
(10) Pub. No.: US 2002/0193780 A1
(43) Pub. Date: Dec. 19, 2002

(54) Bone drilling device

(76) Inventors: Fakhri Karray, Waterloo (CA); Otman Basir, Kitchener (CA); Maher Karray, Tunis (TN); Slaheddine Karray, Manouba (TN); Wayne Pau, Waterloo (CA)

Correspondence Address: CARLSON, GASKEY & OLDS, P.C., 400 WEST MAPLE ROAD, SUITE 350, BIRMINGHAM, MI, 48009, US

(21) Appl. No.: 09/964,717

(22) Filed: Sep. 27, 2001

Related U.S. Application Data
(60) Provisional application No. 60/298,722, filed on Jun. 15, 2001.

(51) Int. Cl. A61B 18/20; A61B 17/72

(52) U.S. Cl. 606/14; 606/79

(57) ABSTRACT

A bone drilling device internally drills holes for the implantation of cross-pins in a fractured bone. An intermedullary nail is surgically implanted into a bone, and an adapter rod, a mounting bracket, and a laser guide rod are assembled on the intermedullary nail. Manual rotation of an exposed internally threaded nut in the mounting bracket engages external threads on the laser guide rod, translating the laser guide rod into the intermedullary nail. A flat face of the laser guide rod contacts a flat portion in the mounting bracket to prevent rotation of the laser guide rod during translation. When the laser guide rod is positioned at the desired location, a laser generates a laser beam which travels down the laser guide rod and reflects off of a mirror at a right angle through a drilling hole in the laser guide rod. The laser beam travels through drilling hole in the intermedullary nail and drills through the bone, creating a hole. A laser diffuser outside the bone defocuses the laser and senses that drilling is complete. Cross-pins are then be inserted into the intermedullary nail and the bone.

This application claims priority to provisional application serial No. 60/298,722 filed Jun. 15, 2001.

### BACKGROUND OF THE INVENTION

The present invention relates generally to a bone drilling device which uses a laser directed through the interior of an intermedullary nail implanted in a fractured bone to drill holes in the bone to allow for the implantation of cross-pins to repair the bone.

Severe bone fractures in long bones commonly have to be surgically repaired to prevent mispositioning during healing. If fractures in long bones do not heal properly, the bone can compress, shortening the length of the bone. Bone fractures in long bones are generally repaired by the use of an intermedullary (IM) nail. After the fractured portions of the bone are re-aligned, an opening is drilled in the bone for insertion of the intermedullary nail in the intermedullary canal. The intermedullary nail is sized to maximize contact with the inner wall of the intermedullary canal.

Holes are drilled perpendicular to the long axis of the bone for the insertion of cross-pins. The drilled holes align with holes in the intermedullary nail to receive the cross-pins, securing the intermedullary nail to the bone. After the bone is healed, the cross-pins and the intermedullary nail are surgically removed from the bone.

The holes in the intermedullary nail are commonly located using x-rays, and an image of the holes is created with a fluoroscopic video. Once the holes in the intermedullary nail are located, holes in the bone are drilled through the exterior of the bone. The drilled holes align with the holes in the intermedullary nail to receive the cross-pin.

There are several drawbacks to the prior bone drilling device. For one, both the surgeon and the patient are exposed to X-rays and radiation from the fluoroscopy in the range of 400 to 1000 rem over the duration of the operation. Over time, prolonged exposure to x-rays can create several health risks.

Therefore, there is a need in the art for an improved bone drilling device which internally drills holes for the implantation of cross-pins in intermedullary nails.

### SUMMARY OF THE INVENTION

This invention relates generally to a bone drilling device which internally drills holes for the implantation of cross-pins in intermedullary nails to repair a fractured bone.

An intermedullary nail including drilling holes is surgically implanted in the intermedullary canal of a fractured bone. An adapter rod sized to fit in the intermedullary nail is inserted into and secured to the intermedullary nail. The adapter rod includes holes which align with the drilling holes of the intermedullary nail. The adapter rod further includes a connector of fixed size.

A mounting bracket is inserted into the connector and receives a laser guide rod. The mounting bracket includes a cutout portion which exposes an internally threaded nut positioned by a spring housed between two Belleville washers. When the nut is manually rotated by a user, the internal threads of the nut engage the external threads on the laser guide rod, translating the laser guide rod into the intermedullary nail. The mounting bracket and laser guide rod further include flat surfaces which contact and prevent rotation of the laser guide rod during translation.

When drilling a hole, the laser guide rod is translated so that a drilling hole in the laser guide rod aligns with the desired drilling hole in the intermedullary nail. When the laser is actuated, a the laser beam travels down the laser guide rod and contacts a mirror which reflects the laser beam out of the laser guide rod drilling hole, through the hole in the intermedullary hole, and into the bone, drilling a hole.

A laser diffuser is positioned outside of the bone drilling area to defocus the laser beam after the laser beam exits the bone. The laser diffuser includes a sensor which senses when the laser beam has cut through the bone. The laser diffuser further includes a cooler to cool the laser diffuser and the drilling area.

After drilling is complete and the bone drilling device is removed, cross-pins are implanted in the holes created by the bone drilling device.

Accordingly, the present invention provides a bone drilling device which internally drills holes for the implantation of cross-pins in intermedullary nails to repair a fractured bone.

These and other features of the present invention will be best understood from the following specification and drawings.

### BRIEF DESCRIPTION OF THE DRAWINGS

The various features and advantages of the invention will become apparent to those skilled in the art from the following detailed description of the currently preferred embodiment. The drawings that accompany the detailed description can be briefly described as follows:

FIG. 1 illustrates a schematic view of a fractured bone with an intermedullary nail inserted into the intermedullary canal;

FIG. 2 illustrates a side view of the bone drilling device of the present invention;

FIG. 3 illustrates a perspective view of the adapter rod shown in FIG. 2;

FIG. 4 illustrates a side view of the mounting bracket shown in FIG. 2;

FIG. 4A illustrates a cross-sectional view of the mounting bracket of FIG. 4 along line 5A-5A;

FIG. 5B illustrates a cross-sectional view of the mounting bracket of FIG. 4 along line 5B-5B;

FIG. 5C illustrates a cross-sectional view of the mounting bracket of FIG. 4 along line 5C-5C;

FIG. 6 illustrates a perspective view of the laser guide rod of FIG. 2;

FIG. 7 illustrates a side view of the laser guide rod of FIG. 2 with the mirror shown in phantom;

FIG. 8 illustrates the laser diffuser; and

FIG. 9 illustrates an exploded view of the laser diffuser;

FIG. 10 illustrates a partial cross-sectional view of a fractured bone with an inserted bone drilling device of FIG. 2.

FIG. 11 illustrates a schematic view of a fractured bone with cross-pins inserted.

### DETAILED DESCRIPTION OF THE PREFERRED EMBODIMENT

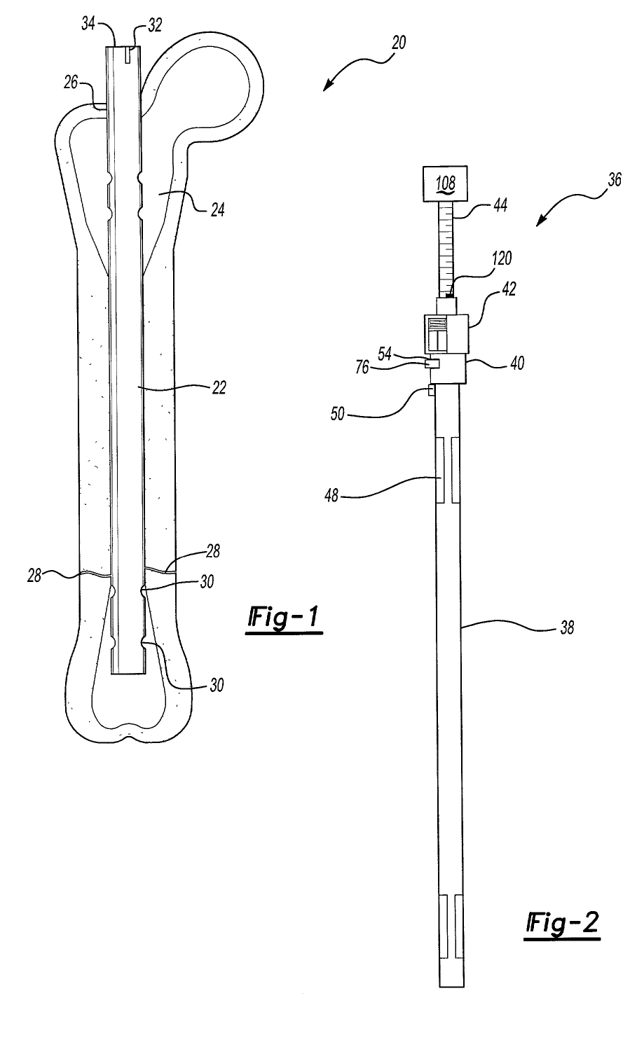

FIG. 1 illustrates a fractured bone 20 with an intermedullary nail 22 surgically implanted into the intermedullary canal 24 through a drilled opening 26 to repair fractures 28. The intermedullary nail 22 is sized to fit in the intermedullary canal 24 such that the intermedullary nail 22 contacts the walls of the intermedullary canal 24 as much as possible. The intermedullary nail 22 includes perpendicular holes 30 through the intermedullary nail 22 positioned at locations where drilling is to occur and cross-pins are to be inserted. A worker skilled in the art would know the proper placement of the holes 30. The intermedullary nail 22 further includes an attachment feature 32 on a first end 34.

FIG. 2 illustrates the bone drilling device 36 of the present invention. The device 36 includes an adapter rod 38 having a connector 40, a mounting bracket 42, and a laser guide rod 44. A laser 108 is attached to the laser guide rod 44.

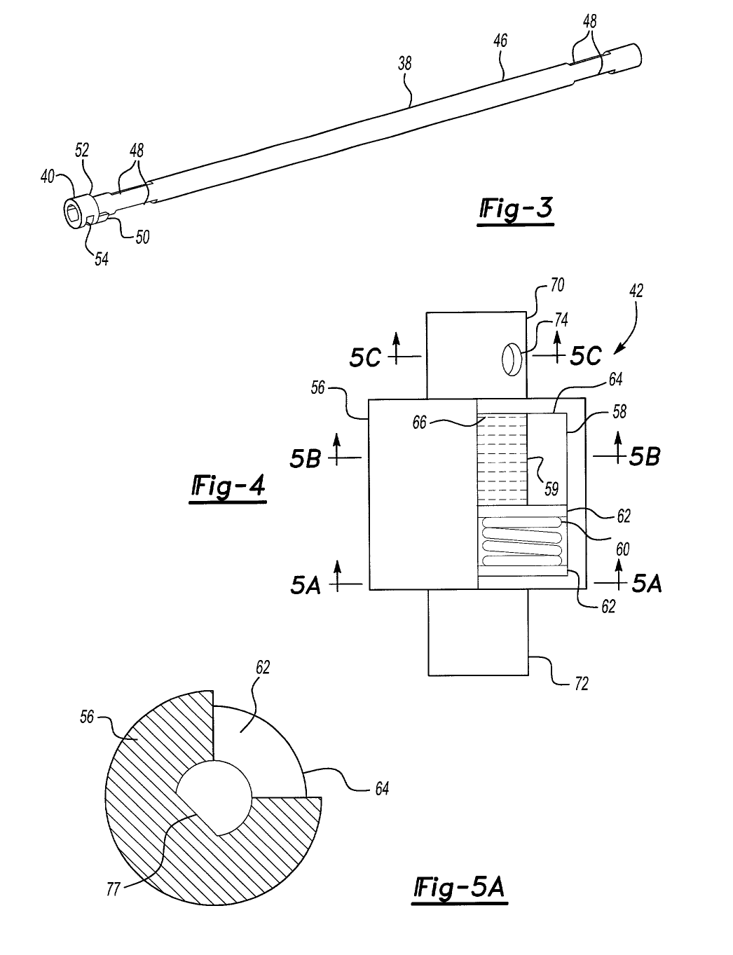

The adapter rod 38, shown in more detail in FIG. 3, includes a tubular body 46 having a plurality of drilling holes 48. The tubular body 46 can be made of different lengths and diameters to be used with intermedullary nails 22 (shown in FIG. 1) of different sizes. An attachment feature 50, preferably a protrusion, is positioned on the first end 52 of the adapter rod 38. The connector 40 attached to the adapter rod 38 is of a fixed size and includes a locking pin hole 54. Adapter rods 38 having tubular bodies 46 of different lengths and diameters all have a standard sized connector 40.

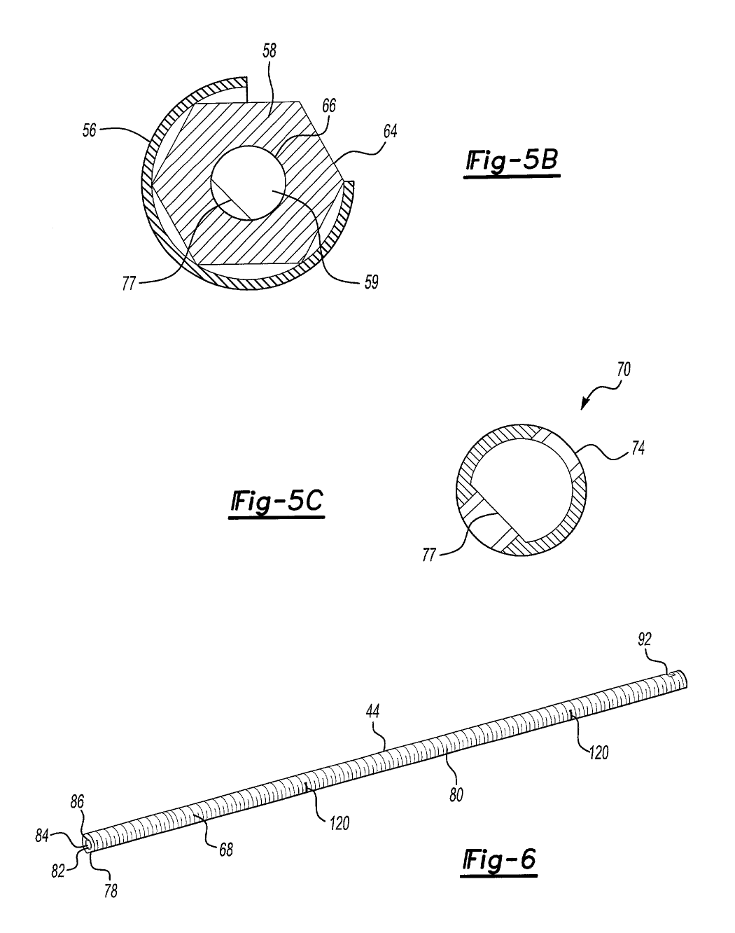

FIG. 4 illustrates the mounting bracket 42 in more detail. The mounting bracket 42 includes an outer casing 56 which houses a nut 58 including an aperture 59 shown in phantom and a plurality of internal threads 66 shown in phantom. A spring 60 positioned between two Belleville washers 62 retains the nut 58 in the desired position. The outer casing 56 includes a cutout portion 64 allowing access to the nut 58. The nut 58 can rotate freely, but cannot move upwards or downwards because of the force supplied by the spring 60. It is preferred that cutout portion 64 be one quarter of the circumference of the outer casing 56. The mounting bracket 42 further includes a first end portion 70 including a locking pin hole 54 to receive a locking pin 76 (shown in FIG. 2) and a second end portion 72. As shown in FIGS. 5A-5C, the outer casing 56 and the end portions 70 and 72 include a flat surface 77. Preferably, the cross-section of the flat portion 77 is a circle with a quarter arc flattened, maintaining 75% of the effective threading surface.

As shown in FIG. 6, the laser guide rod 44 includes an outer surface 80 with a flat face 78 and a plurality of external threads 68 and an inner surface 82 including an inner laser shield 86 which forms a hollow portion 84. The laser shield 86 is made from a laser reflective material to prevent unwanted heat transfer to the laser guide rod 44 and to absorb laser run-off. The laser shield 86 is optimized for the wavelength of the specific laser. The laser guide rod 44 further includes markings 120 to indicate when the laser guide rod 44 is positioned at the desired depth for drilling.

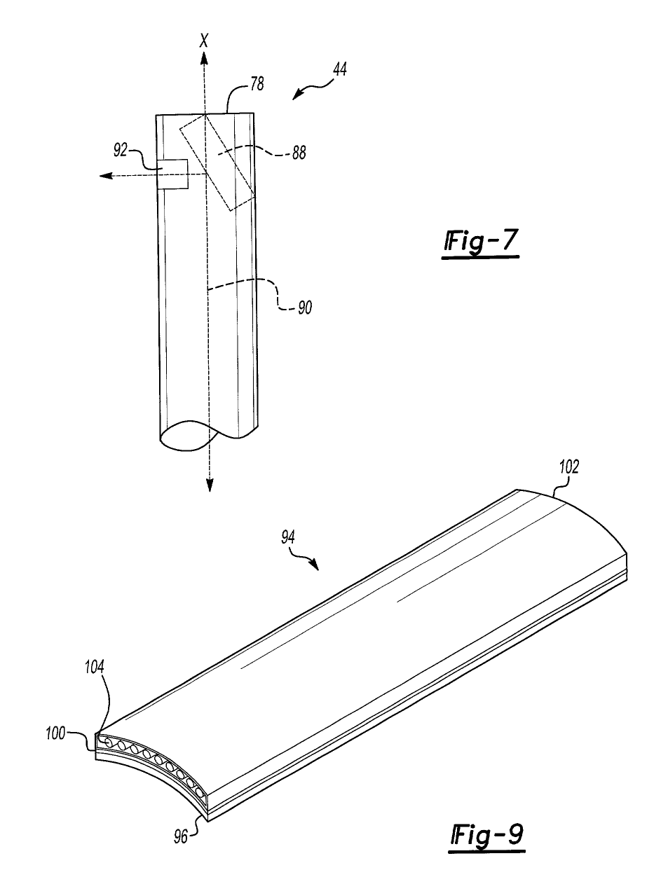

FIG. 7 illustrates a redirection and reflection device 88 positioned in the laser guide rod 44. It is preferred that the reflection device 88 is a high quality optical laser mirror optimized to the specific wavelength of the laser. The laser beam 90 travels down the longitudinal axis x of the laser guide rod 44 and contacts the reflection device 88.

The reflection device 88 directs the laser beam 90 at a right angle from the axis x and out of a drill hole 92 in the laser guide rod 44 and into the bone 20 (shown in FIG. 1). The reflection device 88 is preferably positioned 45 degrees to the axis x of the laser guide rod 44. Alternatively, the reflection device 88 could be a prism.

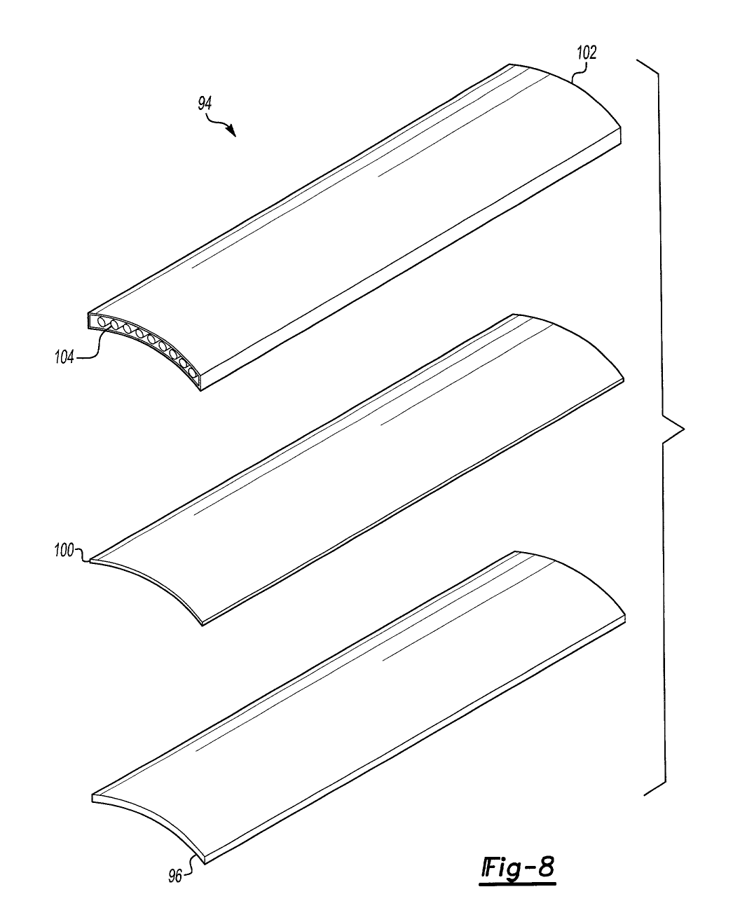

As illustrated in FIGS. 8 and 9, a laser diffuser 94 formed of a heavy metal with a coating absorbs excess energy of the laser beam 90 (shown in FIG. 7). The laser diffuser 94 includes a diffuser array 96, an optical sensor 100, and a cooler 102 including a plurality of tubes 104 containing a cooling fluid.

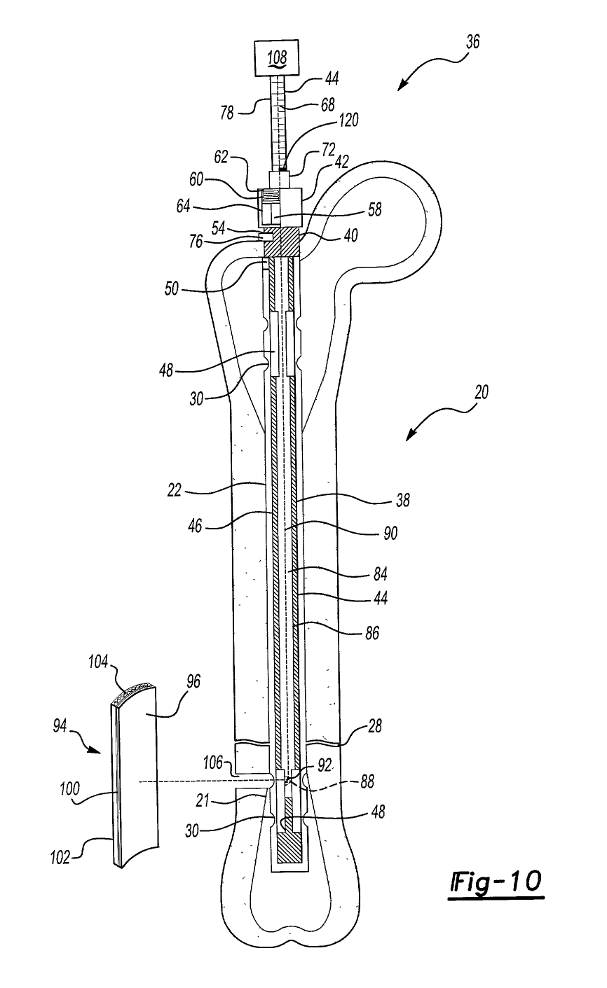

FIG. 10 illustrates the bone drilling device 36 employed in a fractured bone 20. After the intermedullary nail 22 is surgically implanted in a bone 20, the adapter rod 38 is inserted into the intermedullary nail 20 such that the drilling holes 48 of the adapter rod 38 align with the holes 30 of the intermedullary nail 20. The attachment feature 50 of the adapter rod 38 engages the attachment feature 32 (shown in FIG. 1) of the intermedullary nail 22, securing the adapter rod 38 to the intermedullary nail 22. Preferably the attachment feature 50 is a protrusion and the attachment feature 32 is a groove. Alternatively, the attachment feature 50 is a groove and the attachment feature 32 is a protrusion.

The first end portion 70 (shown in FIG. 4) of the mounting bracket 42 is next inserted into the connector 40. As adapter rods 38 having tubular bodies 46 of different lengths and diameters all have a standard sized connector 40, the same mounting bracket 42 can be used for each adapter rod 38 size. The mounting bracket 42 is secured to the connector 40 by inserting a locking pin 76 into the aligned locking pin hole 54 of the connector 40 and the locking pin hole 74 (shown in FIG. 4) of the first end portion 70.

The laser guide rod 44 is received in the second end portion 72 of the mounting bracket 42. The laser guide rod 44 passes through the aperture 59 (shown in FIG. 5B) of the nut 58 and is positioned such that the flat face 78 of the laser guide rod 44 contacts the flat portion 77 (shown in FIG. 5A) of the mounting bracket 46, preventing rotation of the laser guide rod 44. The nut 58 is manually rotated by the user through the cutout portion 64 to translate the laser guide rod 44 into the intermedullary nail 22.

As the nut 58 is rotated, the internal threads 66 (shown in phantom in FIG. 4) of the nut 58 engage the external threads 68 of the laser guide rod 44, translating the laser guide rod 44 into the adapter rod 38 and the intermedullary nail 22. As the nut 58 is rotated, the spring 60 and Bellville washers 62 prevent the nut 58 from translating. The angle of threads 66 (shown in FIG. 4) and 68 control the rate at which the laser guide rod 44 depth is adjusted and prevents longitudinal forces from adjusting the positioning of the laser guide rod 44.

The nut 58 is rotated manually a known number of times until the drill hole 92 of the laser guide rod 44 is aligned with a hole 30 in the intermedullary nail 20. Preferably, the user knows that the drill hole 92 is aligned with a hole 30 by reading markings 120 on the laser guide rod 44, informing the user how far the laser guide rod 44 has translated. The markings on the rod 44 are positioned such that when a marking 120 aligns with the second end 72 of the mounting bracket 42, the drill hole 92 aligns with a hole 30 in the intermedullary nail 20

The laser 108 generates a laser beam 90 which travels down the hollow portion 84 of the laser guide rod 44. If the drill hole 92 is not properly aligned, the inner laser shield 86 will absorb the laser beam 90. The laser guide rod 44 can be repositioned to properly align the drill hole 92 with the hole 30 of the intermedullary nail 22. The laser beam 90 reflects off of the reflection device 88 at a right angle and through the drill hole 92, the drilling hole 48 in the adapter rod 38, through the hole 30 in the intermedullary nail 22, and onto the interior surface 21 of the bone 20. The laser beam 90 drills through the bone 20, creating a hole 106.

After drilling the hole 106, the beam 90 contacts the laser diffuser 94 and is scattered by the diffuser array 96 which defocuses the laser beam 90 and reduces the ability of the laser beam 90 to lath. The diffuser array 96 is partially transparent to the laser beam 90 and curved to defocus the laser beam 90 by reducing the pinpoint focus.

The laser beam 90 then contacts an optical laser sensor 100 which detects the presence of the laser beam 90 and alerts the user that the laser beam 90 has cut through the bone 20. The optical sensor 100 is also curved to increase the contact area. The sensor 100 is tuned to the frequency of the laser beam 90 and is able to absorb large amounts of energy. When the sensor 100 detects the laser beam 90, the bone drilling device 36 is shut off.

The cooler 102 cools the drilling area to reduce heat damage to surrounding tissue. The plurality of tubes 104 in the cooler 102 contain water or a cooling fluid to reduce heat build up in the bone or laser diffuser 94 during the operation.

After the hole 106 is drilled, the nut 58 is manually rotated to translate the laser guide rod 44 and align the drill hole 92 with another selected hole 30 in the intermedullary nail 22. This process is repeated until the desired amount of holes 106 are drilled on both sides of the bone 20. After all the holes 106 are drilled by the bone drilling device 36, the laser 108 is removed. The laser guide rod 44 is removed by reversing the rotation of the nut 58. The locking pin 76 is removed to remove the mounting bracket 42. The adapter rod 38 is removed by disengaging the attachment features 32 and 50.

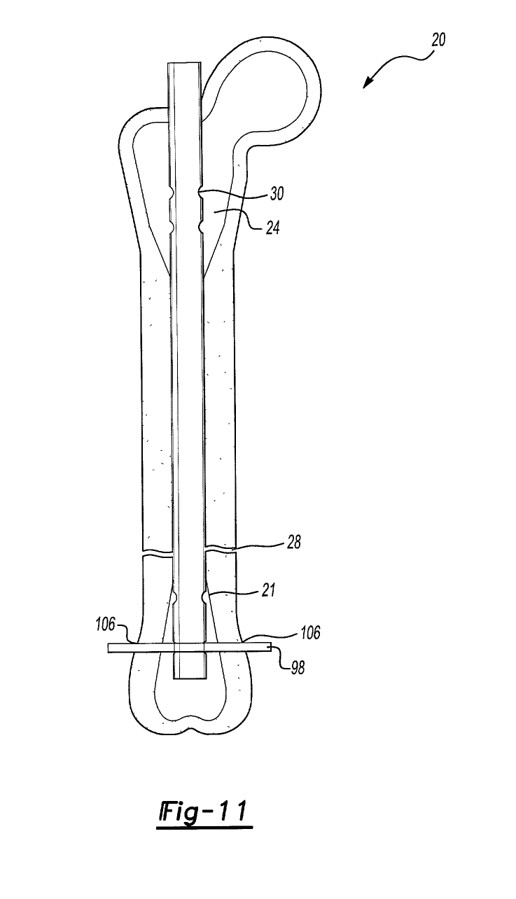

As shown in FIG. 11, the bone drilling device 36 drills holes 106 in a bone 20 for the insertion of cross-pins 98. Once the laser is shut down and the bone drilling device 36 (shown in FIG. 10) is removed, the cross-pin 98 are inserted into the holes 30 to repair the fractured bone 20.

There are several advantages to the bone drilling device 36 of the present invention. For one, the device 36 is simple to use and allows for an exact placement of the holes 106 for insertion of cross-pins 98. There is also a reduction in the exposure of surgeons to X-rays as the holes 106 are drilled internally. As the laser beam 90 is geometrically lined up with the cross-pin hole 30, an accurate hole 106 can be created, reducing error from the location process.

Accordingly, the present invention provides a bone drilling device which internally drills holes for the implantation of cross-pins in intermedullary nails to repair a fractured bone.

The foregoing description is only exemplary of the principles of the invention. Many modifications and variations of the present invention are possible in light of the above teachings. The preferred embodiments of this invention have been disclosed, however, so that one of ordinary skill in the art would recognize that certain modifications would come within the scope of this invention. It is, therefore, to be understood that within the scope of the appended claims, the invention may be practiced otherwise than as specially described. For that reason the following claims should be studied to determine the true scope and content of this invention.

1. A bone drilling device to drill at least one bone hole in a bone comprising:
an intermedullary nail having at least one nail hole; and
a laser beam directed through said at least one nail hole to drill the at least one bone hole from an interior of the bone.

2. The bone drilling device as recited in claim 1 wherein a guide rod is translated in said intermedullary nail until a guide rod drill hole of said guide rod substantially aligns with said at least one nail hole in said intermedullary nail.

3. The bone drilling device as recited in claim 2 wherein said guide rod is mounted to said intermedullary nail by a mounting device.

4. The bone drilling device as recited in claim 3 wherein said mounting device includes a nut having a plurality of internal threads which receives said guide rod having a plurality of external threads, rotation of said nut engages said internal threads of said nut with said external threads of said guide rod to translate said guide rod into said intermedullary nail.

5. The bone drilling device as recited in claim 4 wherein a spring retains said nut in said mounting device.

6. The bone drilling device as recited in claim 3 wherein said mounting device includes a flat portion and said guide rod includes a flat face, the contact of said flat portion and said flat face preventing rotation of said guide rod during translation.

7. The bone drilling device as recited in claim 6 wherein said external threads of said guide rod and said internal threads of said nut maintain approximately 75% contact during rotation of said nut.

8. The bone drilling device as recited in claim 2 wherein said laser beam travels down a longitudinal axis of said guide rod, and a reflective device in said guide rod directs said laser beam substantially perpendicularly to said longitudinal axis and through said guide rod drill hole which is substantially aligned with one of said at least one nail hole of said intermedullary nail and into said bone to drill said bone hole.

9. The bone drilling device as recited in claim 8 wherein said reflective device is substantially 45° to said longitudinal axis of said guide rod.

10. The bone drilling device as recited in claim 1 wherein a laser shield lines an interior surface of said guide rod to absorb said laser beam.

11. The bone drilling device as recited in claim 1 further comprising a laser diffuser positioned proximate to said bone to neutralize said laser beam after drilling said bone hole.

12. The bone drilling device as recited in claim 11 wherein said laser diffuser includes a sensor to detect when said laser beam has drilled said bone hole.

13. The bone drill device as recited in claim 11 wherein said laser diffuser includes a cooler to remove heat in said laser diffuser.

14. The bone drilling device as recited in claim 1 further comprising an adapter to attach said mounting device to said intermedullary nail.

15. The bone drilling device as recited in claim 1 wherein said laser beam is generated by a laser.

16. The bone drilling device as recited in claim 1 wherein a cross-pin is inserted in said bone hole of said bone.

17. A method for drilling a bone hole comprising the step of:
implanting a intermedullary nail having at least one nail hole into an interior of a bone; and
drilling said bone hole in said bone with a laser beam traveling down an interior of said bone.

18. The method as recited in claim 17 further comprising the step of translating a guide rod through said interior of said bone such that a rod guide hole of said guide rod substantially aligns with one of said least one nail hole in said intermedullary nail.

19. The method as recited in claim 17 further comprising the step of defocusing said laser beam after drilling said bone hole.

20. The method as recited in claim 17 wherein the step of translating said guide rod includes manually rotating a nut which receives said guide rod, said nut including a plurality of internal threads which engage a plurality of external threads on said guide rod.

21. A method for drilling a bone hole comprising the step of:
drilling the bone hole from an intermedullary canal of a bone.

22. The method as recited in claim 21 wherein the step of drilling the bone hole includes generating a laser beam and directing the laser beam onto an interior surface of the bone in the intermedullary canal to drill the bone hole through the bone with the laser beam.

23. The method as recited in claim 21 further comprising the step of inserting a cross-pin through the bone hole in the bone to repair a fracture in the bone.
